(19) United States
(12) Patent Application Publication
(10) Pub. No.: US 2020/0289599 A1
(43) Pub. Date: Sep. 17, 2020

(54) A NOVEL CANNABIS PRODUCTION PROCESS AND PRODUCTS THEREOF

(71) Applicant: Curo Medical Ltd., Tel Aviv-Yafo (IL)

(72) Inventor: Joseph MELOUL, Omer (IL)

(21) Appl. No.: 16/753,036

(22) PCT Filed: Oct. 3, 2018
(86) PCT No.: PCT/IL2018/051079

§ 371 Date: Apr. 2, 2020

Related U.S. Application Data
(60) Provisional application No. 62/567,248, filed on Oct. 3, 2017.

(51) Int. Cl. A61K 36/73; B01D 11/02; A61K 31/05; A61K 31/352; A61Q 19/08; A61K 9/00

(52) U.S. Cl. CPC A61K 36/73; B01D 11/0203; B01D 11/0288; A61K 31/05; A61K 31/352; A61K 2236/331; A61K 9/0053; A61K 2236/37; A61K 2236/15; A61K 2236/17; A61K 2236/333; A61Q 19/08

(57) ABSTRACT

The present invention provides a method for producing customizable cannabis products comprising steps of providing preselected plant parts or whole plant; dividing said preselected plant parts or whole plant to a predetermined size distribution; extracting said preselected plant parts or whole plant to provide a first extract; heating said preselected plant parts or whole plant or first extract to a predetermined temperature according to a predetermined time protocol; collecting vapourized material during or after said heating; rendering said vapourized material into a storable intermediate; and formulating said storable intermediate suitable for predetermined administration. The steps of rendering in the form of a storable intermediate comprises reducing the volume of said collected vapourized material to a predetermined volume.

### FIELD OF THE INVENTION

The present disclosure relates to novel method for producing cannabis products. More particularly the current invention pertains to an improved method for producing customized cannabis products having predetermined concentrations of Tetrahydrocannabinol (THC), cannabidiol (CBD), additional cannabinoids, terpenes and derivatives thereof.

### BACKGROUND OF THE INVENTION

There is an unmet long felt need for improved Cannabis production methods and processes to provide consistent and repeatable products customized cannabis products of predetermined active ingredient ratios.

### SUMMARY OF THE INVENTION

It is thus an object of the present invention to provide a method for producing customizable cannabis products comprising steps of:
a. providing preselected plant parts or whole plant
b. dividing said preselected plant parts or whole plant to a predetermined size distribution
c. extracting said preselected plant parts or whole plant to provide a first extract
d. heating said preselected plant parts or whole plant or first extract to a predetermined temperature according to a predetermined time protocol
e. collecting vapourized material during or after said heating
f. rendering said vapourized material into a storable intermediate
g. formulating said storable intermediate suitable for predetermined administration
wherein said steps of rendering in the form of a storable intermediate comprises reducing the volume of said collected vapourized material to a predetermined volume.

It is also an object of the present invention to disclose the aforementioned method, wherein said method comprises steps of combining said storable intermediates of different plant parts, whole plants and/or plant varieties in order to provide a desired composition for use in a predetermined delivery form.

It is also an object of the present invention to disclose the aforementioned method, wherein said method comprises selecting a combination of said storable intermediates of different plant parts, whole plants and/or plant varieties according to intended use of the final cannabis product, mode of delivery of the final cannabis product, treatment indication for the final cannabis product, THC:CBD ratio or other cannabinoid components and/or terpenes and terpenoid components of the aforementioned first extracts of the final cannabis product.

It is also an object of the present invention to disclose the aforementioned method, comprising providing a non-transitory readable medium comprising instructions for selecting combinations and proportions of plant parts, whole plants, trichomes, pistils, cola flowers, sugar leaf, seeds, stems, nodes, fanleafs, stalks, cannabis plant varieties, heating temperature and duration used in steps of said method.

It is also an object of the present invention to disclose the aforementioned method, wherein said steps of providing preselected plant parts comprises selecting trichomes, pistils, cola flowers, sugar leaf, seeds, stems, nodes, fanleafs, stalks in any predetermined proportion.

It is also an object of the present invention to disclose the aforementioned method, wherein said steps of providing preselected plant parts or whole plants comprises selecting whole plants, trichomes, pistils, cola, flowers, sugar leaf, seeds, stems, nodes, fanleafs, stalks, male plants, female plants from any cannabis plant variety in any predetermined proportion.

It is also an object of the present invention to disclose the aforementioned method, wherein said steps of dividing comprises steps selected from grinding, chopping, milling, sieving, filtering, squeezing, kneading, hammering, ultrasonically disrupting, homogenizing, lysing, emulsifying and any other method known in the art.

It is also an object of the present invention to disclose the aforementioned method, wherein said step of extracting is by water extraction, ice-water extraction, alcohol extraction, CO2 extraction, heat pressing, cold pressing or any combination of these or any other method of extraction known in the art.

It is also an object of the present invention to disclose the aforementioned method, wherein said first extract is selected from the group comprising Kief, Dry sieve, dry sift, Hash, Butane Hash Oil, shatter, Budder, honeycomb, crumble, sap, RSO (Rick Simpson Oil), rosin, resin.

It is also an object of the present invention to disclose the aforementioned method, wherein said steps of volume reduction comprises drying, evaporating, filtering and any other method known in the art.

It is also an object of the present invention to disclose the aforementioned method, comprising steps of formulating said storable intermediate into a pharmaceutical product form comprising nanoparticles of size sufficient to passively cross the BBB.

It is also an object of the present invention to disclose the aforementioned method, wherein said steps of formulating comprises formulating said storable intermediate into a configuration selected from the group consisting of lipid based product, cationic liposome, nano emulsion polymer based emulsion, magnetic or magno-electric nanoparticle.

It is also an object of the present invention to disclose the aforementioned method, wherein said steps of formulating comprise configuring said storable intermediate into a pharmaceutically acceptable product form of tablets, liquids, capsules, sustained release capsules or products, sustained release particles, liposome formulations, pills, gels, thin films, rectal suppositories, vaginal suppositories, creams, pastes, ointments, powders, vials, ampoules, cartridges, prefilled syringes.

It is also an object of the present invention to disclose the aforementioned method, wherein said predetermined uses comprise relief of medical indications selected from the group comprising glaucoma, Parkinson, insomnia, Tourette's syndrome.

It is also an object of the present invention to disclose the aforementioned method, wherein said predetermined uses comprise cosmetic uses such as skin treatment and anti-ageing.

It is also an object of the present invention to disclose the aforementioned method, wherein said predetermined uses comprises recreational uses in the form of smoking, vaping, eating, drinking and massaging.

### BRIEF DESCRIPTION OF THE PREFERRED EMBODIMENTS

The novel features believed to be characteristics of the invention are set forth in the appended claims. The invention itself, however, as well as the preferred mode of use, further objects and advantages thereof, will best be understood by reference to the following detailed description of illustrative embodiment when read in conjunction with the accompanying drawings, wherein:

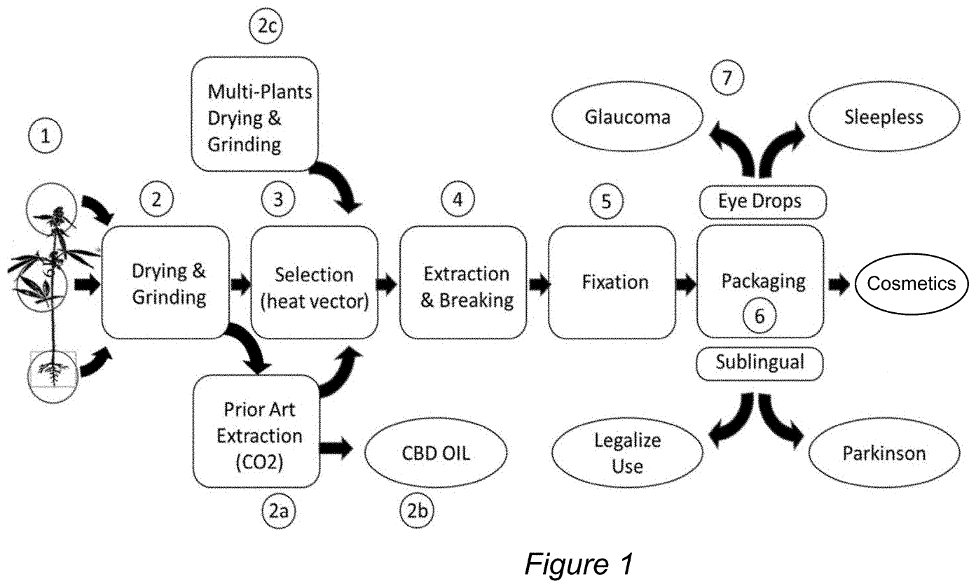

FIG. 1 schematically presents the method of manufacturing cannabis, in accordance with a preferred embodiment of the present invention; and

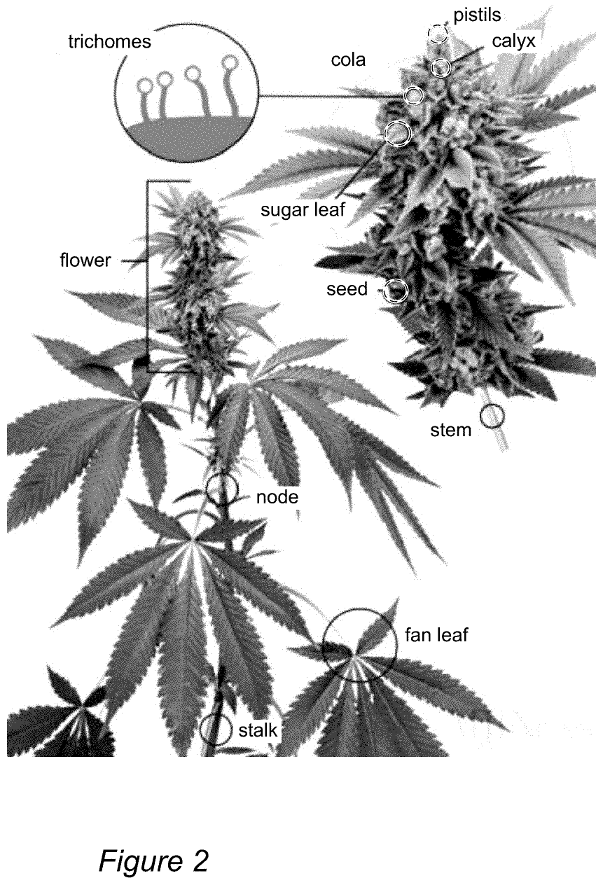

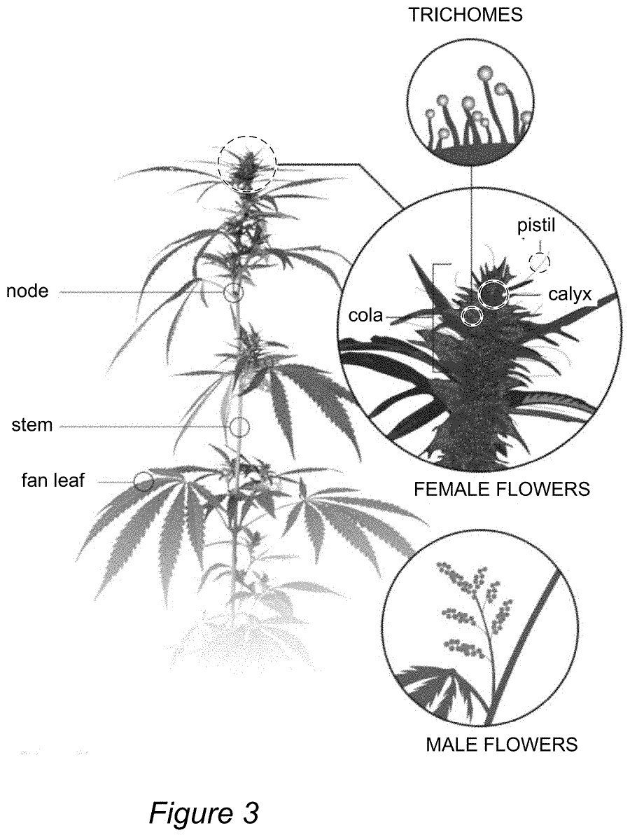

FIGS. 2-3 present the prior art of the cannabis anatomy, in accordance with a preferred embodiment of the present invention.

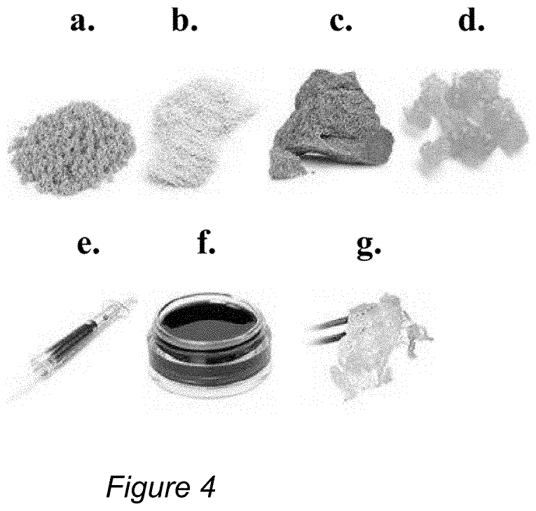

FIGS. 4a-4g present types of cannabis extracts.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured. Reference is now made to FIG. 1 presenting a diagram of the production method of a cannabis based composition for treating a a variety of diseases and disorders such as Glaucoma, sleepless problems, Parkinson or for recreational use.

Reference is now made to FIGS. 2 and 3 are presenting the cannabis anatomy and relevant parts.

The flower is the part of the female cannabis plant that typically has the highest concentration of THC, making it the ideal part of the plant for recreational consumption.

Flowers grow toward the top of the plant and they are commonly referred to as buds.

The cola is the mass of clustered flowers at the top of the cannabis plant.

Trichomes are the tiny resin glands on the flower's leaves and calyces. With the naked eye, they simply look like crystals; under a macroscopic lens, they can look like crystallized mushrooms. Trichomes evolved to protect the cannabis plant from insects and they typically have higher concentrations of THC. Because hashish is made from the resin heads of trichomes, certain strains of cannabis are specifically bred for their high trichome production.

The calyx is a floral structure that forms when the flower begins the budding process; it looks like a tubular sheath surrounding the ovule and pistils. It is composed of sepals, individual plant segments that are often green and occasionally fuzzy. The calyx's trichomes form with their heads pointing toward the top of the calyx.

Pistils are the pollen-catching hairs that extend from the calyx. They can be red, orange or brown in color. They are not known for containing high levels of THC.

The sugar leaf is a small leaf that grows within the flower and it is usually covered in trichomes because of its positioning. Sugar leaves are typically trimmed when flowers are harvested, but their trichome-coating makes them an ideal ingredient when making cannabis edibles.

The cannabis seed is hidden within a flower's calyx. Besides planting a seed to grow a cannabis plant, oil can be extracted from a cannabis seed to be used in human food or animal feed.

The Stems provide the structural support for flowers, while also storing and transporting nutrients for them. Stems aren't known for containing high levels THC, but they can be repurposed for their fiber.

The stalk is the central stem of a plant. As it lacks high levels of THC, it can also be repurposed for its fiber.

The node is the joint where a leaf branches off from the stalk.

The fan leaf is the large leaf that has the photosynthetic responsibility for the cannabis plant. It uses energy from the sun to create chemical energy to feed the plant. Fan leaves lack significant levels of THC, but they have become the unofficial symbol of cannabis.

Reference is herein made to providing preselected plant parts or whole plant (step 1 in FIG. 1).

Reference is herein made to drying, grinding, selecting, extracting and breaking a first extract (step 2, 2a, 2b, 2c, 3, 4 in FIG. 1)

Reference is herein made to steps of dividing said preselected plant parts or whole plant to a predetermined size distribution.

Reference is herein made to steps of extracting said preselected plant parts or whole plant to provide a first extract.

Reference is herein made to steps of heating said preselected plant parts or whole plant or first extract to a predetermined temperature according to a predetermined time protocol

Reference is herein made to steps of collecting vapourized and/or particularized material during or after said heating.

Reference is herein made to steps of rendering said vapourized material and/or particularized into a storable intermediate. This is represented in FIG. 1 by step 5, (fixation). At this fixation step a storable intermediate is provided, of reduced volume relative to the first extract. It is in fact a concentrate of the first extract aforementioned.

Reference is herein made to steps of combining said storable intermediates of different plant parts, whole plants and/or plant varieties in order to provide a desired composition for use in a predetermined delivery form. The predetermined delivery form is selected according to intended use of the final cannabis product, mode of delivery of the final cannabis product, treatment indication for the final cannabis product, THC:CBD ratio or other cannabinoid components and/or terpenes and terpenoid components of the aforementioned first extracts of the final cannabis product.

In the present invention reference is made to first extracts, which may be cannabis concentrates, commonly referred to as cannabis extracts. These are significantly more potent than standard cannabis buds. Their applications as medicine have proven to be effective for patients suffering from all sorts of ailments. When made properly, a cannabis concentrate is reminiscent of the cannabis strain it was extracted from; the smell, taste, and effects are simply magnified due to a larger concentration by weight. Widely used extraction methods are described below in a non-limiting manner.

FIG. 4a-4g Types of Cannabis Extracts

a. Kief

Kief is the simplest of concentrates. Kief is composed of the trichomes (the crystalline structures coating the outside surface of the flowers) broken away from the dried plant material, usually via specialized filtering screens. Kief is generally considered a lower-quality extract, but some top-flight extractors can produce an extremely clean and flavorful product using the dry sieve method. THC content can range from 20 percent to 60 percent.

b. Dry Sieve (Dry Sift)

A popular form of non-solvent hash is dry sieve (sometimes referred to as “dry sift”). Dry sieve is a refined version of kief that has been run through a series of screens so that only the trichome heads remain. The level of quality is often determined by amount of plant matter and capitulate trichome stalks found in the final product. This process at its highest level yields nothing but the largest, most perfect trichome gland heads and none of the gland stems, plant matter, etc. that generally clouds the quicker, lower-quality kief extractions. The most pure dry sieve hash should melt completely when exposed to heat, known as full-melt dry sieve hash.

c. Hash

Ice water extraction is one of the most common processes used to create quality non-solvent hash. The main goal and fundamental idea behind the ice water extraction process is to isolate the trichome heads, which house the essential oils of cannabis, from the stalks and plant matter.

The quality of the resulting hash is often determined by the size of the isolated trichome heads and the extent to which it melts when heated (full-melt being the best). The most important part of the ice water extraction process is drying the final product.

The powdery kief that coats cannabis flowers can be collected and pressed together to form hash. Additionally solvents like ice water or ethanol may be used to more effectively strip the cannabis plant of its cannabinoid-loaded trichomes.

d. Butane Hash Oil (BHO)

Butane Hash Oil, commonly referred to as BHO, is a type of cannabis concentrate made using butane as the main solvent. While a number of variables can determine the final consistency of BHO (mostly temperature), different names when referring to each of the different consistencies of BHO have been applied. Shatter for instance, refers to the glass-like consistency that often snaps or “shatters” when handled. Budder, honeycomb, crumble, and sap are also used to describe the different textures, though they all fall under the category of BHO.

Under this form of extraction, THC content can be as high as 80-90%. This makes BHO a popular choice for many medical marijuana patients suffering from chronic pain, sleep disorders, and other intractable symptoms.

e. Supercritical CO2 Oil

Carbon dioxide (CO2) is a supercritical fluid, meaning it converts into a liquid form when pressurized. At the same time, CO2 is a pure chemical substance that occurs naturally and leaves behind no residues. In fact, supercritical CO2 extraction is already a standard extraction method for the food, dry cleaning and herbal supplement industries. It is a common food additive as well.

The CO2 extraction process allows compounds to be extracted with low toxicity, it utilizes a high pressure vessel containing cannabis. Supercritical CO2 is inserted into the vessel and pumped through a filter where it is separated from the plant matter once the pressure is released. Next, the supercritical CO2 evaporates and is dissolved into the cannabinoids.

f. Rick Simpson Oil (RSO)

Also known as cannabis oil, hemp oil, Phoenix Tears, and Rick Simpson Oil (RSO), whole-plant cannabis oil can be orally administered or applied directly to the skin. Sublingual delivery is the preferred method of treatment for many cancer patients. Not only is it a convenient way to medicate, but intake through the oral mucosal membranes in the mouth provides for rapid and effective absorption directly into the systemic circulation because of the increased bioavailability of the cannabinoids.

Exemplary RSO Small Scale Production Method

Place the completely dry material into a clean bucket.

Dampen the material with the solvent you are using. Many solvents can be used (although it is recommended that you avoid petroleum solvents). You can use ethanol, butane, 99% isopropyl alcohol, or even water. Two gallons of solvent is required to extract the THC from one pound, and 500 ml is enough for an ounce.

Crush the plant material using a stick of clean, untreated wood or any other similar device. Although the material will be damp, it will still be relatively easy to crush up because it is so dry.

Continue to crush the material with the stick, while adding solvent until the plant material is completely covered and soaked. Remain stirring the mixture for about three minutes. As you do this, the THC is dissolved off the material into the solvent.

Pour the solvent oil mixture off the plant material into another bucket. At this point you have stripped the material of about 80% of its THC.

Second wash: again add solvent to the mixture and work for another three minutes to extract the remaining THC.

Pour this solvent oil mix into the bucket containing the first mix that was previously poured out.

Discard the twice washed plant material.

Pour the solvent oil mixture through a coffee filter into a clean container.

Boil the solvent off: a rice cooker will boil the solvent off nicely, and will hold over a half gallon of solvent mixture. CAUTION: avoid stove-tops, red hot elements, sparks, cigarettes, and open flames as the fumes can be extremely flammable.

Add solvent to rice cooker until it is about % full and turn on HIGH heat. Make sure you are in a well-ventilated area and set up a fan to carry the solvent fumes away.

Continue to add mixture to cooker as solvent evaporates until you have added it all to the cooker.

As the level in the rice cooker decreases for the last time, add a few drops of water (about 10 drops of water for a pound of dry material). This will help to release the solvent residue, and protect the oil from too much heat.

When there is about one inch of solvent-water mixture in the rice cooker, swirl the contents until the solvent has finished boiling off.

When the solvent has been boiled off, turn the cooker to LOW heat. At no point should the oil ever reach over 290° F. or 140° C.

Remove the pot containing the oil from the rice cooker. Gently pour the oil into a stainless steel container

Place the stainless steel container in a dehydrator, or put it on a gentle heating device such as a coffee warmer. It may take a few hours but the water and volatile terpenes will be evaporated from the oil. When there is no longer any surface activity on the oil, it is ready for use.

Suck the oil up in a plastic syringe, or in any other container you see fit. A syringe will make the oil easy to dispense. When the oil cools completely it should have the consistency of thick grease.

g. Rosin

Rosin has been gaining a lot of traction in the medical cannabis community as of lately and for good reason. Rosin is a solid form of resin that is obtained by adding pressure & heat to vaporize volatile liquid terpenes, typically with an industrial heat press (or even a hair straightener for small batches).

The rosin technique is quick, simple and affordable, allowing anyone to create quality solventless hash in a matter of seconds. To get started making Rosin, you only need a few basic tools in order to create a quality finished product, but not nearly as many as you need with other extraction techniques.

It is herein disclosed an improved method for producing customized cannabis products having predetermined concentrations of Tetrahydrocannabinol (THC), cannabidiol (CBD), additional cannabinoids, terpenes and traces and derivatives thereof.

It is herein disclosed a pharmaceutically acceptable product in the form of caramel-like edible product comprising a cannabis derived formulation sufficient to reduce blood pressure in a hypertensive human subject by approximately 20 mmg. (diastolic or systolic) within approximately 10-15 mins of consumption.

It is an object of the present invention to disclose a method for producing customizable cannabis products comprising steps of:
a. providing preselected plant parts or whole plant
b. dividing said preselected plant parts or whole plant to a predetermined size distribution
c. extracting said preselected plant parts or whole plant to provide a first extract
d. heating said preselected plant parts or whole plant or first extract to a predetermined temperature according to a predetermined time protocol
e. collecting vapourized material during or after said heating
f. rendering said vapourized material into a storable intermediate
g. formulating said storable intermediate suitable for predetermined administration

In preferred embodiments the steps of rendering in the form of a storable intermediate comprises reducing the volume of said collected vapourized and/or particularized material to a predetermined volume.

In preferred embodiments the method comprises steps of combining said storable intermediates of different plant parts, whole plants and/or plant varieties in order to provide a desired composition for use in a predetermined delivery form.

In preferred embodiments the method comprises selecting a combination of said storable intermediates of different plant parts, whole plants and/or plant varieties according to intended use of the final cannabis product, mode of delivery of the final cannabis product, treatment indication for the final cannabis product, THC:CBD ratio or other cannabinoid components and/or terpenes and terpenoid components of the aforementioned first extracts of the final cannabis product.

In preferred embodiments, the method comprising providing a non-transitory readable medium comprising instructions for selecting combinations and proportions of plant parts, whole plants, trichomes, pistils, cola flowers, sugar leaf, seeds, stems, nodes, fanleafs, stalks, cannabis plant varieties, heating temperature and duration used in steps of said method.

In preferred embodiments the steps of providing preselected plant parts comprises selecting trichomes, pistils, cola flowers, sugar leaf, seeds, stems, nodes, fanleafs, stalks in any predetermined proportion.

In preferred embodiments the steps of providing preselected plant parts or whole plants comprises selecting whole plants, trichomes, pistils, cola, flowers, sugar leaf, seeds, stems, nodes, fanleafs, stalks, male plants, female plants from any cannabis plant variety in any predetermined proportion.

In preferred embodiments the steps of dividing comprises steps selected from grinding, chopping, milling, sieving, filtering, squeezing, kneading, hammering, ultrasonically disrupting, homogenizing, lysing, emulsifying and any other method known in the art.

In preferred embodiments the step of extracting is by water extraction, ice-water extraction, alcohol extraction, CO2 extraction, heat pressing, cold pressing or any combination of these or any other method of extraction known in the art.

In preferred embodiments the first extract is selected from the group comprising Kief, Dry sieve, dry sift, Hash, Butane Hash Oil, shatter, Budder, honeycomb, crumble, sap, RSO (Rick Simpson Oil), rosin, resin

In preferred embodiments the steps of volume reduction comprises drying, evaporating, filtering and any other method known in the art.

It is a further embodiment of the present invention to provide the present method comprising steps of water capture of water soluble particles and optionally cycling through a filter membrane and further water capturing volumes so as to provide particle size of approximately 50 nm.

In preferred embodiments, comprising steps of formulating said storable intermediate into a pharmaceutical product form comprising nanoparticles of size sufficient to passively cross the BBB.

In preferred embodiments the steps of formulating comprises formulating said storable intermediate into a configuration selected from the group consisting of lipid based product, water soluble product, and/or vapourized or particularized particles cationic liposome, nano emulsion polymer based emulsion, magnetic or magno-electric nanoparticle.

In preferred embodiments the steps of formulating comprise configuring said storable intermediate into a pharmaceutically acceptable product form of tablets, liquids, capsules, sustained release capsules or products, sustained release particles, liposome formulations, pills, gels, thin films, rectal suppositories, vaginal suppositories, creams, pastes, ointments, powders, vials, ampoules, cartridges, prefilled syringes.

In preferred embodiments the predetermined uses comprise relief of medical indications selected from the group comprising glaucoma, Parkinson, insomnia, Tourette's syndrome.

In preferred embodiments the predetermined uses comprise cosmetic uses such as skin treatment and anti-ageing.

In preferred embodiments the predetermined uses comprises recreational uses in the form of smoking, vaping, eating, drinking and massaging.

1. A method for producing customizable cannabis products comprising steps of:
a. providing preselected plant parts or whole plant;
b. dividing said preselected plant parts or whole plant to a predetermined size distribution;
c. extracting said preselected plant parts or whole plant to provide a first extract;
d. heating said preselected plant parts or whole plant or first extract to a predetermined temperature according to a predetermined time protocol;
e. collecting vapourized and/or particularized material during or after said heating;
f. rendering said vapourized and/or particularized material into a storable intermediate; and,
g. formulating said storable intermediate suitable for predetermined administration; wherein said steps of rendering in the form of a storable intermediate comprises reducing the volume of said collected vapourized and/or particularized material to a predetermined volume and concentration suitable for a pharmaceutically acceptable product.

2. The method of claim 1, wherein said method comprises steps of combining said storable intermediates of different plant parts, whole plants and/or plant varieties in order to provide a desired composition for use in a predetermined delivery form.

3. The method of claim 1, wherein said method comprises selecting a combination of said storable intermediates of different plant parts, whole plants and/or plant varieties according to intended use of the final cannabis product, mode of delivery of the final cannabis product, treatment indication for the final cannabis product, THC:CBD ratio or other cannabinoid components, trace components and/or terpenes and terpenoid components of the aforementioned first extracts of the final cannabis product.

4. The method of claim 1, comprising providing a non-transitory readable medium comprising instructions for selecting combinations and proportions of plant parts, whole plants, trichomes, pistils, cola flowers, sugar leaf, seeds, stems, nodes, fanleafs, stalks, cannabis plant varieties, heating temperature and duration used in steps of said method.

5. The method of claim 1, wherein said steps of providing preselected plant parts comprises selecting trichomes, pistils, cola flowers, sugar leaf, seeds, stems, nodes, fanleafs, stalks in any predetermined proportion.

6. The method of claim 1, wherein said steps of providing preselected plant parts or whole plants comprises selecting whole plants, trichomes, pistils, cola, flowers, sugar leaf, seeds, stems, nodes, fanleafs, stalks, male plants, female plants from any cannabis plant variety in any predetermined proportion.

7. The method of claim 1, wherein said steps of dividing comprises steps selected from grinding, chopping, milling, sieving, filtering, squeezing, kneading, hammering, ultrasonically disrupting, homogenizing, lysing, emulsifying and any other method known in the art.

8. The method of claim 1, wherein said step of extracting is by water extraction, ice water extraction, alcohol extraction, CO2 extraction, heat pressing, cold pressing or any combination of these or any other method of extraction known in the art.

9. The method according to claim 1 wherein said first extract is selected from the group comprising Kief, Dry sieve, dry sift, Hash, Butane Hash Oil, shatter, Budder, honeycomb, crumble, sap, RSO (Rick Simpson Oil), rosin, resin.

10. The method of claim 1, wherein said steps of volume reduction comprises drying, evaporating, filtering and any other method known in the art.

11. The method of claim 1, wherein said steps of volume reduction comprises water capture of water soluble particles and optionally cycling through a filter membrane and further water capturing volumes so as to provide particle size of approximately 50 nm.

12. The method of claim 1, comprising steps of formulating said storable intermediate into a pharmaceutical product form comprising nanoparticles of size sufficient to passively cross the BBB.

13. The method of claim 12, wherein said steps of formulating comprises formulating said storable intermediate into a configuration selected from the group consisting of lipid based product, cationic liposome, nano emulsion polymer based emulsion, magnetic or magno-electric nanoparticle.

14. The method of claim 1, wherein said steps of formulating comprise configuring said storable intermediate into a pharmaceutically acceptable product form of tablets, liquids, capsules, sustained release capsules or products, sustained release particles, liposome formulations, pills, gels, thin films, rectal suppositories, vaginal suppositories, creams, pastes, ointments, powders, vials, ampoules, cartridges, prefilled syringes.

15. The method of claim 1, wherein said predetermined uses comprise relief of medical indications selected from the group comprising glaucoma, Parkinson, insomnia, Tourette's syndrome.

16. The method of claim 1, wherein said predetermined uses comprises production of eyedrops for glaucoma treatment.

17. The method of claim 1, wherein said predetermined uses comprise cosmetic uses such as skin treatment and anti-ageing.

18. The method of claim 1, wherein said predetermined uses comprises recreational uses in the form of smoking, vaping, eating, drinking and massaging.

19. The method of claim 1 wherein said pharmaceutically acceptable product is a caramel-like edible product comprising a cannabis derived formulation sufficient to reduce blood pressure in a hypertensive human subject by approximately 20 mmg. (diastolic or systolic) within approximately 10-15 mins of consumption.

20. A cannabis product comprising at least one storable intermediate of the method of claim 1, wherein said product has a predetermined volume and concentration suitable for a pharmaceutically acceptable product.
